(19) United States
(12) Patent Application Publication
(10) Pub. No.: US 2021/0382328 A1
(43) Pub. Date: Dec. 9, 2021

(54) Transparent Body

(71) Applicant: Nippon Telegraph and Telephone Corporation, Tokyo (JP)

(72) Inventors: Yoko Ono, Musashino-shi, Tokyo (JP); Hironobu Minowa, Musashino-shi, Tokyo (JP); Shuhei Sakamoto, Musashino-shi, Tokyo (JP); Takeshi Komatsu, Musashino-shi, Tokyo (JP)

(21) Appl. No.: 17/287,221

(22) PCT Filed: Nov. 6, 2019
(86) PCT No.: PCT/JP2019/043351

§ 371 Date: Apr. 21, 2021

(30) Foreign Application Priority Data
Nov. 20, 2018 (JP) 2018-216968

(51) Int. Cl. G02C 11/08; H01M 50/133; H01M 50/124; H01M 50/531; H01M 10/0525

(52) U.S. Cl. CPC G02C 11/08; H01M 50/133; H01M 2220/30; H01M 50/531; H01M 10/0525; H01M 50/124

(57) ABSTRACT

A battery is appropriately housed in a transparent body. A transparent body includes: a transparent body main part; a defogger that removes fog on the transparent body main part; and a battery that supplies electric power to the defogger. The battery includes: a positive electrode where a transparent positive electrode conductive film and a transparent positive electrode layer are laminated on an insulating transparent positive electrode side housing; a negative electrode where a transparent negative electrode conductive film and a transparent negative electrode layer are laminated on an insulating transparent negative electrode side housing; and a transparent electrolyte layer that is arranged between the positive electrode layer and the negative electrode layer which face each other. A film thickness of each of the positive electrode conductive film, the negative electrode conductive film, the positive electrode layer, and the negative electrode layer is such a thickness as to prevent absorption of visible light of incident light to promote transmission.

### TECHNICAL FIELD

The present invention relates to a transparent body having a defogger.

### BACKGROUND ART

For transparent bodies having transparency such as glass and lens, a defogger is sometimes used so as to avoid poor visibility due to condensation. For example, when goggles are used in a low-temperature environment such as a skiing area, a temperature difference, which is caused when air in the goggles warmed by heat from skin of a human body contacts a lens which has been cooled by an outside air, causes condensation and fog formation.

In recent years, goggles with a defogger that removes formed condensation have been increasingly used. As a defogging method, a method of replacing air within goggles by an electric fan, a method of vaporizing condensation adhered to goggles by heat generation from heating wire, and the like are provided. Thus, a generally used defogger is driven by electric power.

Electric power used for the defogger of goggles is supplied by a portable battery. The battery is housed in a battery housing part provided at a temple part of a frame or in a wearer's clothing pocket or the like by being connected from the goggles by cable (see Non-Patent Literature 1).

### CITATION LIST

### Non-Patent Literature

Non-Patent Literature 1: SWANS HELI-XED BK Surface heat generating lens goggles, [online], [searched on Oct. 19, 2018], internet<URL: https://netshop.swans.co.jp/shopdetail/000000000899/>

### SUMMARY OF THE INVENTION

### Technical Problem

However, there is no method for properly housing a battery in a transparent body. For example, in housing a battery at an edge of a goggles frame, there may occur a problem with a goggles design. In addition, in connecting a battery from goggles by cable and housing it in a pocket, there also may occur a problem with design and wearer's movability.

In supplying electric power to a defogger used for a transparent body as described above, it is sometimes difficult to properly house a battery without compromising transparency in some cases.

Therefore, it is an object of the present invention to provide a transparent body capable of properly housing a battery.

### Means for Solving the Problem

In order to solve the above problems, the features of the present invention relate to a transparent body having a defogger. The transparent body according to the features of the present invention includes: a transparent body main part; a defogger that removes fog on the transparent body main part; and a battery that supplies electric power to the defogger. The battery includes: a positive electrode where a transparent positive electrode conductive film and a transparent positive electrode layer are laminated on an insulating transparent positive electrode side housing; a negative electrode where a transparent negative electrode conductive film and a transparent negative electrode layer are laminated on an insulating transparent negative electrode side housing; and a transparent electrolyte layer that is arranged between the positive electrode layer and the negative electrode layer which face each other. A film thickness of each of the positive electrode conductive film, the negative electrode conductive film, the positive electrode layer, and the negative electrode layer is such a thickness as to prevent absorption of visible light of incident light to promote transmission.

The transparent body main part includes a first transparent layer and a second transparent layer. The first transparent layer and the second transparent layer may be provided so as to face each other. The battery may be provided between the first transparent layer and the second transparent layer.

The positive electrode layer and the negative electrode layer may be formed so as to allow lithium ion insertion and removal.

The positive electrode conductive film and the negative electrode conductive film may be formed so as to have a film thickness of 100 nm or more and 500 nm or less; and may be made of a semiconductor of tin-doped indium oxide, tin oxide, fluorine-doped tin oxide, or zinc oxide.

The electrolyte layer may have lithium-ion conductivity.

A first collector tab where the positive electrode conductive film is exposed and a second collector tab where the negative electrode conductive film is exposed may be provided; and the first collector tab and the second collector tab may be connected to the defogger.

### Effects of the Invention

According to the present invention, the transparent body capable of properly housing a battery can be provided.

### BRIEF DESCRIPTION OF DRAWINGS

FIG. 1 is a diagram that describes the overview of a transparent body according to an embodiment of the present invention.

FIG. 2 is a diagram that describes goggles using the transparent body according to the embodiment of the present invention.

FIG. 3 is a diagram that describes a cross section of the transparent body according to the embodiment of the present invention.

FIG. 4 is a diagram that describes a battery used for the transparent body according to the embodiment of the present invention.

### DESCRIPTION OF EMBODIMENTS

Next, an embodiment of the present invention will be described with reference to drawings. In the following description of the drawings, the same or similar components are denoted by the same or similar reference signs.

Transparent Body

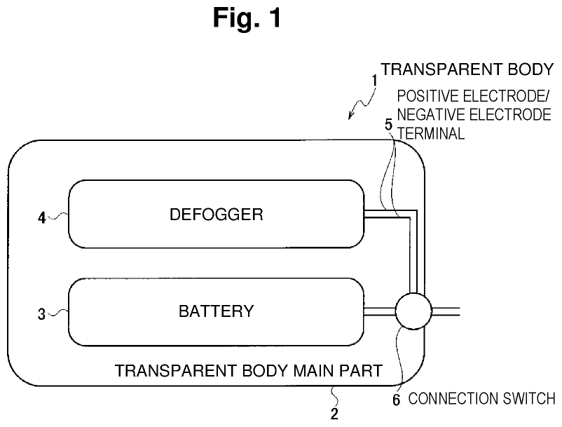

A transparent body 1 having a defogger according to the embodiment of the present invention includes, as shown in FIG. 1, a transparent body main part 2, a battery 3, a defogger 4, a positive electrode/negative electrode terminal 5, and a connection switch 6. In the embodiment of the present invention, “transparent” means a property of an object capable of preventing the absorption and reflection of incident light and does not mean emission of all of the incident light as outgoing light.

The transparent body main part 2 is a transparent object. For the transparent body main part 2, an optimum material is appropriately used according to a function of an object for which the transparent body 1 is used. For example, when the transparent body 1 according to the embodiment of the present invention is used for ski goggles, the transparent body main part 2 is a transparent lens. When the transparent body 1 according to the embodiment of the present invention is used for a window, the transparent body main part 2 is transparent glass.

The battery 3 is a transparent sheet-shaped battery. The battery 3 supplies electric power to the defogger 4. It is used by overlapping with the whole or part of the transparent body main part 2, according to a power storage capacity of the battery 3 and specifications of a product for which the transparent body 1 is used. The battery 3 will be described in detail later.

The defogger 4 is driven by electric power supplied from the battery 3 and removes fog on the transparent body main part 2. In the embodiment of the present invention, a mechanism of the defogger 4 does not matter. As a defogging mechanism, for example, a method of replacing air around the transparent body main part 2 by an electric fan, a method of vaporizing condensation adhered to the transparent body main part 2 by heat generation from heating wire such as nichrome wire, or a method of vaporizing condensation adhered to the transparent body main part by causing a film surface to generate heat through the energization of a transparent conductive film is available.

The positive electrode/negative electrode terminal 5 connects the battery 3 and the defogger 4 to supply electric power of the battery 3 to the defogger 4. The positive electrode/negative electrode terminal 5 may be used also for a charging operation of the battery 3.

The connection switch 6 allows connection or disconnection of the positive electrode/negative electrode terminal 5 to be externally operated. The connection switch 6 controls connection between the battery 3 and the defogger 4, thereby allowing control of driving or stop of the defogger 4 to be controlled.

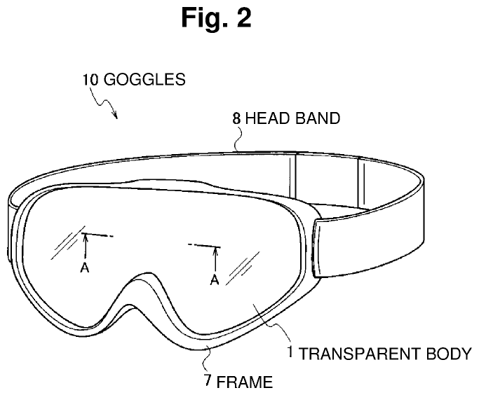

With reference to FIG. 2, an example of using the transparent body 1 according to the embodiment of the present invention for goggles 10 will be presented. The goggles 10 include the transparent body 1, a frame 7, and a head band. The transparent body 1 is held by the frame 7. To the frame 7, a head band 8 is connected. The head band 8 is composed of a member having elasticity and has a length adjusting function so that it can be tightly attached to a head part. By having the head band 8 attached to the head part, the goggles 10 is worn.

The transparent body 1 used for the goggles 10 covers the eyes of a wearer. Where a skin side of the transparent body 1 is warmed by heat coming from the skin of the wearer and condensation occurs, the condensation is eliminated by the defogger 4 driven by the battery 3.

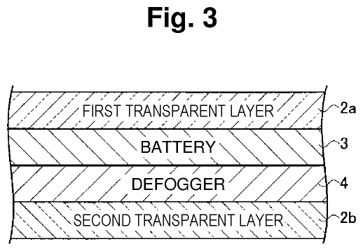

With reference to FIG. 3, a cross section of the transparent body 1 will be described. FIG. 3 is a view showing a cross section A-A′ of FIG. 2 when viewed from below. The transparent body main part 2 of the transparent body 1 shown in FIG. 3 includes a first transparent layer 2a and a second transparent layer 2b. The first transparent layer 2a and the second transparent layer 2b are provided so as to face each other. The first transparent layer 2a is positioned at an opposite side (outer side) of the wearer when the wearer wears the goggles 10; and the second transparent layer 2b is positioned on a wearer's side.

For the first transparent layer 2a and the second transparent layer 2b, their materials do not matter as long as they are those used for goggles lens; however, they are preferable to be polycarbonate resins having high shock resistance and flexibility and also having heat resistance to heating by the defogger 4.

The battery 3 and the defogger 4 are provided between the first transparent layer 2a and the second transparent layer 2b. In an example shown in FIG. 3, the defogger 4 is arranged on a side of the second transparent layer 2b and the battery 3 is arranged on a side of the first transparent layer 2a. The second transparent layer 2b is positioned on a skin side and in comparison with the first transparent layer 2a, easily causes condensation to occur. By arranging the defogger 4 on the second transparent layer 2b side, the occurrence of condensation on the second transparent layer 2b can be reduced.

Although an example shown in FIG. 3 shows that the defogger 4 is formed over the entire surfaces of the first transparent layer 2a and the second transparent layer 2b, it is not limited thereto. For example, it is only required that a target to be defogged by the defogger 4 is between the second transparent layer 2b and the battery 3.

For example, when fog is removed by replacing air by an electric fan, the defogger 4 replaces air in a space between the second transparent layer 2b and the battery 3.

When fog is removed by vaporizing condensation by heat from heating wire, the heating wire is provided between the second transparent layer 2b and the battery 3 to warm a space between the second transparent layer 2b and the battery 3. As for the heating wire, its arrangement position does not matter as long as a space between the second transparent layer 2b and the battery 3 is warmed. The heating wire may be provided at a position that does not obstruct visibility, for example, on left and right end sides of the frame 7. In addition, when the heating wire has color or thickness that does not affect view, it may be arranged widely on a surface of the transparent body 1.

When fog is removed by causing a film surface to generate heat through energization of the transparent conductive film, the transparent conductive film is provided between the second transparent layer 2b and the battery 3.

The cross section of the transparent body 1 shown in FIG. 3 is one example and is not limited thereto. For example, the transparent body main part 2 may be formed so as to expose the battery 3 or the defogger 4 without forming a plurality of layers.

Battery

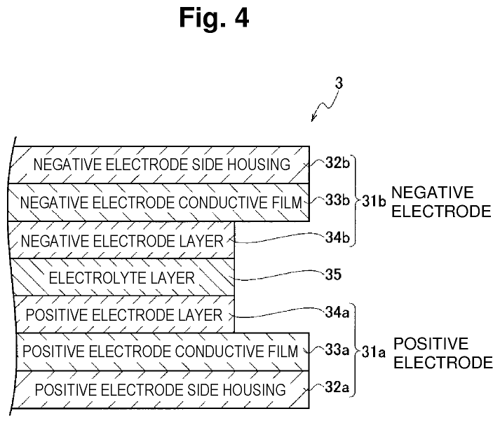

The transparent battery 3 according to the embodiment of the present invention will be described with reference to FIG. 4. In the lens (transparent body main part 2) used for the goggles 10, as a goggles lens generally has a curved surface and a sphere surface, the battery 3 is formed so flexible as to be able to match with the shape of a lens surface.

The battery 3 according to the embodiment of the present invention includes a positive electrode 31a, a negative electrode 31b, and an electrolyte layer 35.

The positive electrode 31a is formed by laminating a transparent positive electrode conductive film 33a and a transparent positive electrode layer 34a on an insulating transparent positive electrode side housing 32a. The negative electrode 31b is formed by laminating a transparent negative electrode conductive film 33b and a transparent negative electrode layer 34b on an insulating transparent negative electrode side housing 32b. The electrolyte layer 35 is transparently formed and is arranged between the positive electrode layer 34a and the negative electrode layer 34b which face each other. A film thickness of each of the positive electrode conductive film 33a, the negative electrode conductive film 33b, the positive electrode layer 34a, and the negative electrode layer 34b is such a thickness as to prevent absorption of visible light of incident light to promote transmission.

The battery 3 utilizes the principle of a known lithium second battery. In the embodiment of the present invention, a case of utilizing the principle of a lithium ion secondary battery among the lithium secondary batteries will be described.

Each of the positive electrode side housing 32a and the negative electrode side housing 32b is a transparent flexible film such as a polyethylene terephthalate (PET) film.

Each of the positive electrode layer 34a and the negative electrode layer 34b is formed so as to allow lithium ion insertion and removal. The electrolyte layer 35 has a lithium ion conductivity. The positive electrode layer 34a and the negative electrode layer 34b are arranged so as to face each other via the electrolyte layer 35 to prevent contacting each other.

The positive electrode conductive film 33a is formed on the positive electrode side housing 32a by a sputtering method, a vapor deposition method, or a spin coating method. Similarly, the negative electrode conductive film 33b is formed on the negative electrode side housing 32b by a sputtering method, a vapor deposition method, or a spin coating method.

Each of the positive electrode conductive film 33a and the negative electrode conductive film 33b has a film thickness of 100 nm or more and 500 nm or less. The positive electrode conductive film 33a and the negative electrode conductive film 33b are made of a semiconductor of tin-doped indium oxide (ITO), tin oxide (TO), fluorine-doped tin oxide (FTC), zinc oxide (ZnO), or the like. It is preferable that each of the positive electrode conductive film 33a and the negative electrode conductive film 33b is formed by a sputtering method to have a film thickness of 100 nm or more and 200 nm or less in consideration of light transparency.

The positive electrode layer 34a is obtained by forming, on the positive electrode conductive film 33a, a material allowing lithium ion insertion and removal by a sputtering method, a vapor deposition method, or a spin coating method. The negative electrode layer 34b is obtained by forming, on the negative electrode conductive film 33b, a material allowing lithium ion insertion and removal by a sputtering method, a vapor deposition method, or a spin coating method. As for a film thickness of each of the positive electrode layer 34a and the negative electrode layer 34b, it is desirable that the film thickness is smaller in consideration of light transparency; and it is desirable that the each of them is formed by a sputtering method within a range from 100 nm to 500 nm in which a charge/discharge capacity can be obtained. Unevenness on surfaces of the positive electrode layer 34a and the negative electrode layer 34b can be reduced by a sputtering method and reflection of incident light can be further prevented.

For the positive electrode layer 34a, an oxide such as lithium cobalt oxide (LiCoO₂), lithium manganate oxide (LiMn₂O₄), lithium iron phosphate (LiFePO₄), lithium nickel oxide (LiNiO₂), or lithium titanate (LiTi₂O₄, Li₄Ti₅O₁₂), which is thinly deposited so that light absorption is suppressed to allow light transmission, can be used. For the negative electrode layer 34b, an oxide such as lithium titanate (LiTi₂O₄, Li₄Ti₅O₁₂), titanium oxide (TiO₂), zinc oxide (ZnO), tin oxide (TO), indium oxide (In₂O₃), tin-doped indium oxide (ITO), or fluorine-doped tin oxide (FTC)) can be used. A combination of respective materials is selected so that the potential of the negative electrode layer 34b is lower than that of the positive electrode layer 34a. In order to obtain high transmittance, it is desirable to use Li₄Ti₅O₁₂ for the positive electrode layer 34a and In₂O₃ for the negative electrode layer 34b.

On a part of an upper surface of each of the positive electrode conductive film 33a and the negative electrode conductive film 33b, the positive electrode layer 34a or the negative electrode layer 34b is formed. On the positive electrode conductive film 33a and the negative electrode conductive film 33b, parts where the positive electrode layer 34a and the negative electrode layer 34b are not formed, are exposed as collector tabs. On a part where the positive electrode conductive film 33a is exposed, a first collector tab is formed; and a second collector tab where the negative electrode conductive film 33b is exposed is formed. The first collector tab a and the second collector tab b are connected to the defogger 4 via the positive electrode/negative electrode terminal 5.

For the electrolyte layer 35, a solid electrolyte, a polymer electrolyte, or the like which transmits visible light, among the conventional solid electrolytes containing lithium ions can be used. The electrolyte layer 35 is formed so as to contact both a surface on a side of the negative electrode layer 34b of the positive electrode layer 34a and a surface on a side of the positive electrode layer 34a of the negative electrode layer 34b.

A method for forming the battery 3 will be described.

First, two pieces of PET film, which is 0.1 mm thick, 100 mm long, and 250 mm wide, are prepared as the positive electrode side housing 32a and the negative electrode side housing 32b. On the entire surface of one side of each of them, an ITO film deposited by a sputtering method using an ITO target is formed as the positive electrode conductive film 33a and the negative electrode conductive film 33b.

The positive electrode layer 34a and the negative electrode layer 34b are deposited by a sputtering method on parts of respective surfaces of the positive electrode conductive film 33a and the negative electrode conductive film 33b which are ITO films. The film thickness of both the positive electrode layer 34a and the negative electrode layer 34b is set to 200 nm. By masking an end of 100×10 mm of a surface region of 100×250 mm of each of the positive electrode conductive film 33a and the negative electrode conductive film 33b which are ITO films, the positive electrode layer 34a or the negative electrode layer 34b is formed in a remaining surface region of 100×240 mm of each of the ITO films. On the positive electrode conductive film 33a and the negative electrode conductive film 33b, parts where the positive electrode layer 34a and the negative electrode layer 34b is not formed are exposed as collector tabs.

The positive electrode layer 34a and the negative electrode layer 34b are an Li₄Ti₅O₁₂ film and an In₂O₃ film, respectively, which are formed by a sputtering method using Li₄Ti₅O₁₂ and In₂O₃ targets, respectively.

Each of the positive electrode 31a and the negative electrode 31b which are thus obtained is processed into a shape accommodated in the frame 7 of the goggles 10. As the electrolyte layer 35, a solution is generated in which polyvinylidene fluoride (PVDF) powder as a binder, an organic electrolyte solution prepared by dissolving 1 mol/L of lithium bis(trifluoro-methanesulfonyl) imide (LiTFSi) as lithium salt in propylene carbonate (PC), and N-methyl-2-pyrrolidone (NMP) as a dispersion medium are mixed at a weight ratio of 1:9:10. The generated solution is stirred at 60° C. for one hour in dry air at or below a dew point of −50° C.; 50 ml of the solution is poured into a petri dish of 200 Φ; and is dried at 50° C. for 12 hours. As a result, a transparent film with a thickness of 1 μm is formed.

The electrolyte layer 35 thus prepared is formed into the shape of regions of the positive electrode layer 34a and the negative electrode layer 34b and is sandwiched so that the positive electrode layer 34a of Li₄Ti₅O₁₂ and the negative electrode layer 34b of In₂O₃ face each other and so that only a deposition surface is entirely covered. In addition, sandwiching is performed by a commercially available transparent film for laminator which has a thickness of 100 μm so that only the collector tabs of the positive electrode and the negative electrode are exposed to an outside; and hot pressing is entirely performed at 130° C., thereby forming the battery 3. The transmittance of the entire battery 3 is 70% or higher in a visible light region. The battery thus formed was charged to 3 V at room temperature by using a commercially available charge/discharge measurement system.

When the battery 3 formed as described above was connected to a heating film, it was confirmed that heat was generated. In addition, using polycarbonate resins as the first transparent layer 2a and the second transparent layer 2b, the battery 3 and the defogger 4 are sandwiched so as to be closely attached by these two layers, thereby forming the transparent body 1 having a configuration of FIG. 3.

Lastly, testing for a defogging effect was performed. Spraying was performed to a side of the second transparent layer 2b of the transparent body 1 by using a spray, to artificially generate fog; and the defogger 4 was driven by using the battery 3 within the transparent body 1. As a result, it was confirmed that the fog was removed after 10 minutes.

The battery 3 according to the embodiment of the present invention as described above is formed to be sheet-shaped and to be transparent; and therefore, allows a defogging function to be added without deteriorating the design and wearing feeling of goggles. In addition, by providing the transparent battery 3 in the transparent body 1 such as a lens, anti-fog goggles which does not cause the battery to obstruct visibility can be provided. Furthermore, since the battery 3 is a chargeable/dischargeable secondary battery, it can also be repeatedly used.

As described above, the battery 3 according to the embodiment of the present invention can be appropriately housed within the transparent body 1.

Other Applications

The transparent body with a defogging function, which is described in the embodiment of the present invention, may be applied to an object made of glass, such as window glass, car window. Since the battery 3 according to the embodiment of the present invention is flexibly formed, it can be easily stuck to a flat surface such as glass.

### Other Embodiments

As described above, description has been made according to the embodiment of the present invention. However, it should not be understood that the description and drawings constituting a part of this disclosure limit the present invention. From this disclosure, various alternative embodiments, examples, and operational techniques will be apparent to those skilled in the art.

The present invention naturally includes various embodiments not described herein. Therefore, the technical scope of the present invention is defined only by the matters specifying the invention according to the scope of claims reasonable from the above description.

### REFERENCE SIGNS LIST

1 Transparent body

2 Transparent body main part

2a First transparent layer

2b Second transparent layer

3 Battery

4 Defogger

5 Positive electrode/negative electrode terminal

6 Connection switch

7 Frame

8 Head band

10 Goggles

31a Positive electrode

31b Negative electrode

32a Positive electrode side housing

32b Negative electrode side housing

33a Positive electrode conductive film

33b Negative electrode conductive film

34a Positive electrode layer

34b Negative electrode layer

35 Electrolyte layer

1. A transparent body having a defogger, comprising:
a transparent body main part;
the defogger that removes fog on the transparent body main part; and
a battery that supplies electric power to the defogger,
wherein the battery includes:
a positive electrode where a transparent positive electrode conductive film and a transparent positive electrode layer are laminated on an insulating transparent positive electrode side housing;
a negative electrode where a transparent negative electrode conductive film and a transparent negative electrode layer are laminated on an insulating transparent negative electrode side housing; and
a transparent electrolyte layer that is arranged between the positive electrode layer and the negative electrode layer which face each other, and
a film thickness of each of the positive electrode conductive film, the negative electrode conductive film, the positive electrode layer, and the negative electrode layer is such a thickness as to prevent absorption of visible light of incident light to promote transmission.

2. The transparent body according to claim 1, wherein
the transparent body main part includes a first transparent layer and a second transparent layer,
the first transparent layer and the second transparent layer are provided so as to face each other, and
the battery is provided between the first transparent layer and the second transparent layer.

3. The transparent body according to claim 1, wherein the positive electrode layer and the negative electrode layer are formed so as to allow lithium ion insertion and removal.

4. The transparent body according claim 1, wherein the positive electrode conductive film and the negative electrode conductive film are formed to have a film thickness of 100 nm or more and 500 nm or less; and are made of a semiconductor of tin-doped indium oxide, tin oxide, fluorine-doped tin oxide, or zinc oxide.

5. The transparent body according to claim 1, wherein the electrolyte layer has a lithium ion conductivity.

6. The transparent body according to claim 1, comprising:
a first collector tab where the positive electrode conductive film is exposed; and
a second collector tab where the negative electrode conductive film is exposed,
wherein the first collector tab and the second collector tab are connected to the defogger.

7. The transparent body according to claim 2, wherein the positive electrode layer and the negative electrode layer are formed so as to allow lithium ion insertion and removal.

8. The transparent body according to claim 2, wherein the positive electrode conductive film and the negative electrode conductive film are formed to have a film thickness of 100 nm or more and 500 nm or less; and are made of a semiconductor of tin-doped indium oxide, tin oxide, fluorine-doped tin oxide, or zinc oxide.

9. The transparent body according to claim 3, wherein the positive electrode conductive film and the negative electrode conductive film are formed to have a film thickness of 100 nm or more and 500 nm or less; and are made of a semiconductor of tin-doped indium oxide, tin oxide, fluorine-doped tin oxide, or zinc oxide.

10. The transparent body according to claim 2, wherein the electrolyte layer has a lithium ion conductivity.

11. The transparent body according to claim 3, wherein the electrolyte layer has a lithium ion conductivity.

12. The transparent body according to claim 4, wherein the electrolyte layer has a lithium ion conductivity.

13. The transparent body according to claim 2, comprising:
a first collector tab where the positive electrode conductive film is exposed; and
a second collector tab where the negative electrode conductive film is exposed,
wherein the first collector tab and the second collector tab are connected to the defogger.

14. The transparent body according to claim 3, comprising:
a first collector tab where the positive electrode conductive film is exposed; and
a second collector tab where the negative electrode conductive film is exposed,
wherein the first collector tab and the second collector tab are connected to the defogger.

15. The transparent body according to claim 4, comprising:
a first collector tab where the positive electrode conductive film is exposed; and
a second collector tab where the negative electrode conductive film is exposed,
wherein the first collector tab and the second collector tab are connected to the defogger.

16. The transparent body according to claim 5, comprising:
a first collector tab where the positive electrode conductive film is exposed; and
a second collector tab where the negative electrode conductive film is exposed,
wherein the first collector tab and the second collector tab are connected to the defogger.
